(19) United States
(12) Patent Application Publication
(10) Pub. No.: US 2008/0044473 A1
(43) Pub. Date: Feb. 21, 2008

(54) METHODS OF REDUCING NERVOUS SYSTEM GLIAL SCAR FORMATION USING CYCLOSPORINE COMPONENTS

(76) Inventor: Milan Radojicic, Santa Rosa, CA (US)

Correspondence Address: Milan Radojicic, 4145 Shadow Lane, Apt. 435, Santa Rosa, CA, 95405, US

(21) Appl. No.: 11/840,199

(22) Filed: Aug. 16, 2007

Related U.S. Application Data
(60) Provisional application No. 60/822,640, filed on Aug. 17, 2006.

(51) Int. Cl. A61K 38/13; A61K 9/10; A61P 25/00

(52) U.S. Cl. 424/484; 514/11

(57) ABSTRACT

This invention relates to methods of treating mammals at risk or affected by central nervous glial scarring by administering a cyclosporine component. Among the conditions treated are, but not limited to, epidural scarring, meningeal fibrosis, arachnoiditis, nerve root tethering, brain and spinal cord injury, as well as peripheral nerve, tendon and ligament scarring.

### CROSS-REFERENCE TO RELATED APPLICATIONS

This application claims the benefit of provisional patent application Ser. No. 60/822,640, filed Aug. 17, 2006 by the present inventor.

### FEDERALLY SPONSORED RESEARCH

Not applicable.

### SEQUENCE LISTING OR PROGRAM

Not applicable.

### BACKGROUND OF THE INVENTION

1. Field of the Invention

The field of the present invention relates to methods of providing intended therapeutic effects to mammals using cyclosporine components. In particular, the invention relates to methods of administering therapeutically effective amounts of a cyclosporine component to a mammal affected or at risk for nervous system glial scar formation for the purposes of producing a therapeutic affect.

2. Prior Art

In surgery, injury and disease of the central and peripheral nervous system tissues, abnormal scar tissue formation may form in the vicinity of the brain, spinal cord, dural sac, as well the peripheral nerves and tendons and ligaments. This glial scar is identified differently according to the location of the insult, but generally results from a local inflammatory reaction triggering the growth and proliferation of glial cells. While intended as a wound healing mechanism, this process of glial scarring may at time go awry and result in untoward effects.

When glial scarring is located above the dural tissue, this is called an epidural scar. This epidural scar may thicken and cause compression on the dural sac and the brain and spinal cord, as well brain and spinal nerves and nerve roots. When the scar forms within the dura, this condition is called meningeal fibrosis or arachnoiditis. Arachnoiditis may cause alterations in cerebrospinal fluid flow and dynamics, as well as edema and pressure on the brain and spinal cord. Glial scar may also form within the substance of the brain and spinal cord. Glial scarring within the brain and spinal cord may inhibit axonal regeneration and lead to irregular patterns of electrical activity in the brain. Glial scarring may also lead to dural and nerve root tethering, as well as scarring of the peripheral nerves and tendons and ligaments. In general, this scarring may lead to significant morbidity and mortality in patients.

Significant work has been devoted to identifying means of preventing this nervous system glial scar. However, no single method has yielded consistent results.

The use of cyclosporine components for therapeutics in other tissues has been the subject of various patents, for example Stern et al. U.S. Pat. No. 7,151,085. However, until this Applicant's invention, no such method has been applied to preventing or reducing nervous system glial scar formation. This invention uses a cyclosporine component administered in a therapeutically active dose for the purposes of preventing or reducing nervous system glial scar formation.

### DETAILED DESCRIPTION OF THE INVENTION

This invention relates to methods of treating mammals at risk or affected by central nervous glial scarring by administering a cyclosporine component. Among the conditions treated are, but not limited to, epidural scarring, meningeal fibrosis, arachnoiditis, nerve root tethering, brain and spinal cord injury and peripheral nerve and tendon and ligament scarring.

In the preferred embodiment, the administering step comprises topical administration of the cyclosporine component to an area of the nervous at risk for the development of a glial scar, such as the exposed meninges immediately after a laminectomy operation. Topical administration allows a therapeutically active amount of the cyclosporine component to be delivered locally without substantial systemic absorption and side effects. The topical administration of the cyclosporine may be in form of, but not limited to, a biodegradable polymer, hydrogel or emulsion.

In another embodiment, the administering step comprises the mucosal or parenteral administration of the cyclosporine component to an area of the nervous system undergoing a glial scar, such as a patient with recent spinal cord injury who is not hemodynamically stable for operative intervention. In this instance, the therapeutic benefits of the cyclosporine component may outweigh the consequences of systemic side effects.

Cyclosporin is an immunosuppressant drug thought to bind to the cytosolic protein cyclophilin of T-lymphocytes. This complex of cyclosporin and cyclophylin inhibits calcineurin, which under normal circumstances is responsible for activating the transcription of interleukin-2. Interleukin-2 (IL-2) is a hormone of the immune system that is instrumental in the body's natural response to microbial infection and in discriminating between foreign (nonself) and self. Cyclosporine also inhibits lymphokine production and interleukin release and therefore leads to a reduced function of effector T-cells. These aforementioned properties allow the mitigation of the inflammatory response responsible for the uncontrolled nervous system glial scar formation that leads to untoward effects. Any suitable cyclosporine component effective in the present methods may be used.

### EXAMPLE 1

A male patient, age 24, suffers a fractured cervical vertebrae and spinal cord injury. After laminectomy and surgical fixation, a cyclosporine component contained within a hydrogel is applied extradurally to the exposed meninges and nerve roots. Such treatment reduces both intradural, dural, and extradural glial activation and hence glial scar formation. After such treatment and at one, two and six months post-application, the patient's pain levels, as well as motor and sensory scores are better than untreated controls.

### EXAMPLE 2

A female patient, age 54, undergoes a lumbar laminectomy and foraminotomy for spinal stenosis. The surgeon places a cyclosporine component contained within a biodegradable polymer/wafer within the recesses of the laminectomy in order to reduce epidural scarring and nerve root scarring. At one and six months post-surgery, the patient reports no pain and an MRI with contrast shows no evidence of dural scarring.

### EXAMPLE 3

A male patient, age 34, undergoes repair of a torn flexor tendon of his right middle finger. The surgeon places a biodegradable polymer containing a cyclosporine component in the vicinity of the repair site. Post-operatively and at two and six months post-surgery, the patient reports excellent mobility of the involved digit and no pain.

### EXAMPLE 4

A male patient, age 27, with a Chiari malformation and cervical syrinx undergoes a surgical procedure which involves a duraplasty and detethering of arachnoid granulations. The surgeon places a cyclosporine component contained within a hydrogel extradurally in order to prevent recurrences of the arachnoiditis. Post-operatively and at two and six months, the patient is symptom free and an MRI at six months post-surgery shows no residual syrinx, normal CSF flow and no evidence of dural scarring.

### EXAMPLE 5

A paraplegic female patient, age 34, who suffered a spinal cord injury at the thoracic level at age 18 is noted to have an expanded syrinx at the thoracic level and she is symptomatic.

While the above description contains many specificities, these should not be construed as limitations on the scope of the invention, but as exemplifications of the presently preferred embodiments thereof. Many other ramifications and variations are possible within the teaching of the invention. Additionally, any combination of the above examples may be possible.

Thus the scope of the invention should be determined by the appended claims and their legal equivalents, rather than the examples given.

1. A method for reducing glial scar formation in the central and peripheral nervous system of a mammal comprising administering of a cyclosporine component selected from the group consisting of cyclosporin A, salts thereof and mixtures, to said human or animal in an amount sufficient to reduce glial scar formation.

2. The method of claim 1 wherein the cyclosporine component is cyclosporinee A.

3. The method of claim 1, wherein the administration of the compound is accomplished by a method selected from the group consisting of topical, transdermal topical, intraoperative topical, mucosal, injection, intramuscular injection, intravenous injection, implantation infusion device and inhalation.

4. The method of claim 1 wherein the cyclosporine component is administered in an emulsion.

5. The method of claim 1 wherein the cyclosporine component is administered in a hydrogel.

6. The method of claim 1 wherein the cyclosporine component is administered in a biodegradable biopolymer.

7. The method of claim 4 wherein the emulsion includes about 0.01 to 15% by weight of the cyclosporine component.

8. The method of claim 5 wherein the hydrogel includes about 0.01% to about 50% by weight of the cyclosporine component.

9. The method of claim 6 wherein the biodegradable biopolymer includes about 0.01% to about 40% by weight of the cyclosporine component.
